(19) United States
(12) Patent Application Publication
(10) Pub. No.: US 2026/0098048 A1
(43) Pub. Date: Apr. 9, 2026

(54) IMPROVED PROCESS FOR THE PREPARATION OF RISDIPLAM AND ITS INTERMEDIATES

(71) Applicant: Natco Pharma Limited, Hyderabad (IN)

(72) Inventors: Sathaiah KANDULA, Hyderabad (IN); Rama Krishna YADLAPALLI, Hyderabad (IN); Bulli Babi VIPPARLA, Hyderabad (IN); Sathish THUMATI, Hyderabad (IN); Sreenivas NAGAPURI, Hyderabad (IN); Srikanth PARAKALA, Hyderabad (IN); Sashikanth SUTHRAPU, Hyderabad (IN); Ramesh DANDALA, Hyderabad (IN); Pulla Reddy MUDDASANI, Hyderabad (IN); Venkaiah Chowdary NANNAPANENI, Hyderabad (IN)

(21) Appl. No.: 19/113,864

(22) PCT Filed: Sep. 25, 2023
(86) PCT No.: PCT/IN2023/050885

§ 371 Date: Mar. 20, 2025

(30) Foreign Application Priority Data
Sep. 26, 2022 (IN) 202241055182

(51) Int. Cl. C07D 519/00; C07D 241/38

(52) U.S. Cl. CPC C07D 519/00; C07D 241/38

(57) ABSTRACT

The present invention relates to an improved process for the preparation of Risdiplam compound of Formula-I. The present invention also relates to novel intermediates of Risdiplam compound of Formula-I and process for the preparation of the same.

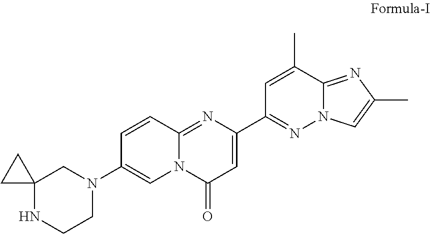 [SMILES: Cc1cn2nc(-c3cc(=O)n4cc(N5CCNC6(CC6)C5)ccc4n3)cc(C)c2n1]

### FIELD OF THE INVENTION

The present invention relates to an improved process for the preparation of Risdiplam. The present invention also relates to novel intermediates of Risdiplam and process for the preparation of the same.

### BACKGROUND OF THE INVENTION

Risdiplam is a survival of motor neuron-2 (SMN2) gene splicing modifier indicated for the treatment of spinal muscular atrophy (SMA). The chemical name of Risdiplam is 7-(4,7-diazaspiro[2.5]octan-7-yl)-2-(2,8-dimethylimidazo[1,2-b]pyridazine-6-yl)-pyrido-4H-[1,2-a]pyrimidin-4-one. Risdiplam can be represented by the following chemical structure according to Formula (I).

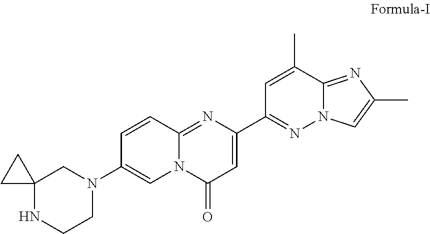 [SMILES: Cc1cn2nc(-c3cc(=O)n4cc(N5CCNC6(CC6)C5)ccc4n3)cc(C)c2n1]

Risdiplam was approved by the US Food and Drug Administration (FDA) in August 2020, for the treatment of spinal muscular atrophy (SMA) in pediatric and adult patients, sold under the brand name Evrysdi®.

U.S. Pat. No. 9,969,754 discloses a process for the preparation of Risdiplam compound of Formula-I as illustrated in the Scheme-I below. First, the compound of Formula-II is treated with ammonium hydroxide or aqueous ammonia (25%) to obtain mixture of compounds of Formula-III and Formula-IIIa. The obtained mixture is further reacted with 1-bromo-2,2-dimethoxypropane in the presence of Pyridinium p-toluenesulfonate (PPTS) in 2-propanol to obtain compound of Formula-IV, which upon reaction with 4,4,4′,4′,5,5,5′,5′-octamethyl-2,2′-bi(1,3,2-dioxaborolane) in the presence of Pd(dppf)Cl₂·CH₂Cl₂ and KOAc in dioxane to obtain compound of Formula-V. Coupling of compound of Formula-V with compound of Formula-VI in the presence of Pd(PPh₃)₄ and aq. K₂CO₃ in acetonitrile to obtain compound of Formula-VII, which is further reacted with compound of Formula-VIII in the presence of DIPEA in DMSO and isolated the Risdiplam compound of Formula-I by using column chromatography with 18% of yield.

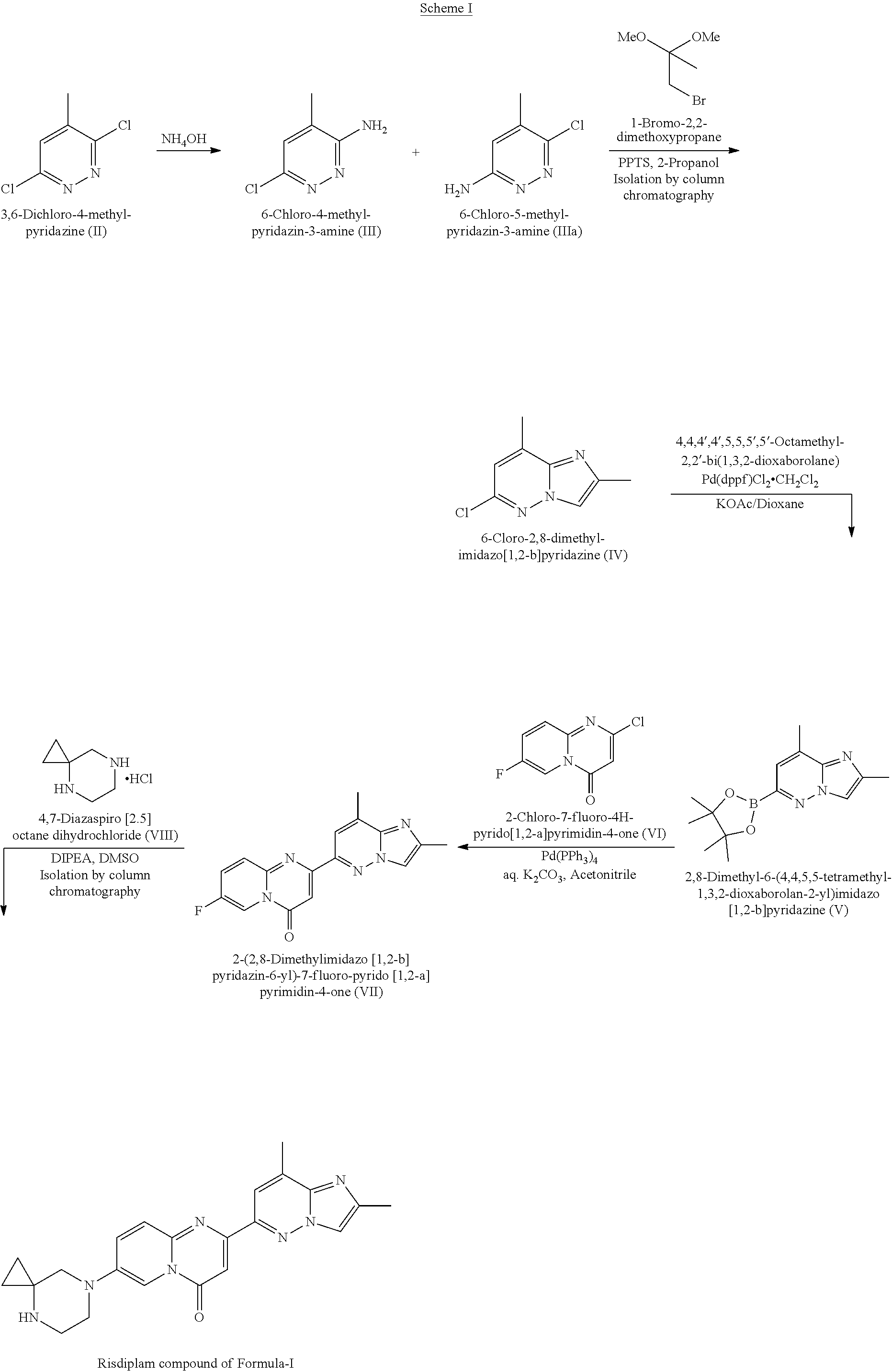

There is still need for an improved, cost-effective, commercially scalable and environment friendly process for the preparation of Risdiplam.

### SUMMARY OF THE INVENTION

The present invention provides a process for the preparation of Risdiplam compound of Formula-I,

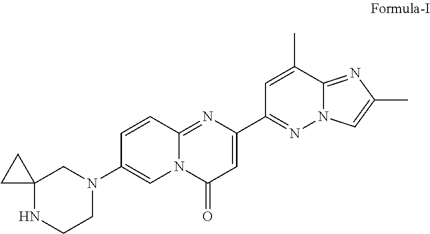 [SMILES: Cc1cn2nc(-c3cc(=O)n4cc(N5CCNC6(CC6)C5)ccc4n3)cc(C)c2n1]

comprising the steps of:
a) reacting a compound of Formula-VII,

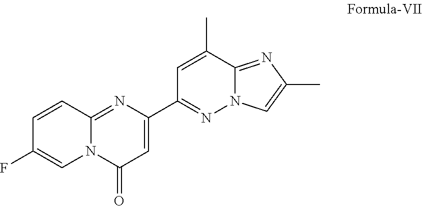 [SMILES: Cc1cn2nc(-c3cc(=O)n4cc(F)ccc4n3)cc(C)c2n1]

with a compound of Formula-IX

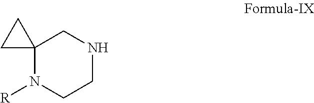

wherein, R is amine protecting group other than Boc (tert-butoxy carbonyl); in presence of a base in a suitable solvent to obtain compound of Formula-X

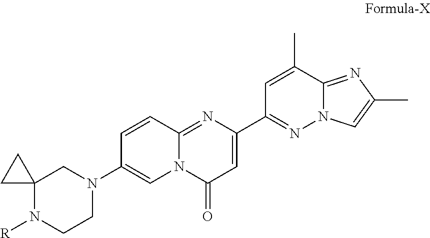

b) deprotecting the compound of Formula-X in presence of an acid in a suitable solvent followed by reaction with oxalic acid dihydrate to obtain Risdiplam oxalic acid salt compound of Formula-Ia.

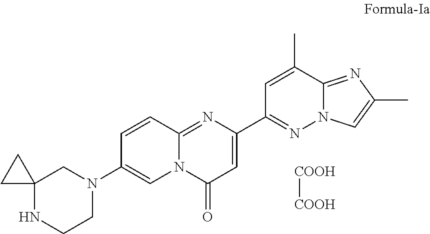

c) treating the compound of Formula-Ia with an acid followed by base in a suitable solvent to obtain Risdiplam compound of Formula-I.

The present invention further provides a process for the preparation of compound of Formula-VII,

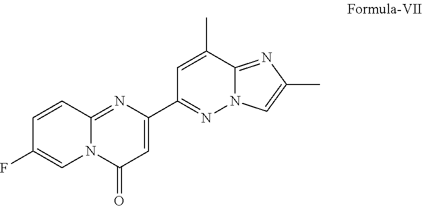 [SMILES: Cc1cn2nc(-c3cc(=O)n4cc(F)ccc4n3)cc(C)c2n1]

comprising the steps of:
a) reacting a compound of Formula-III

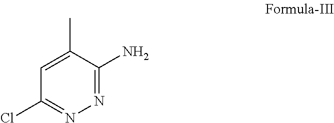

with 1-bromo-2,2-dimethoxypropane in the presence of p-Toluenesulfonic acid monohydrate in a suitable solvent to obtain compound of Formula-IV,

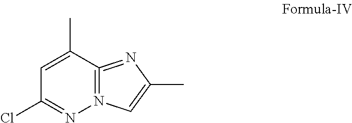

b) treating the compound of Formula-IV with bis(pinacolato)diboron in the presence of a base and catalyst in a suitable solvent to obtain compound of Formula-V,

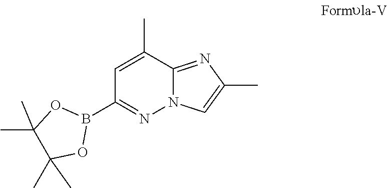

c) reacting the compound of Formula-V in situ with compound of Formula-VI

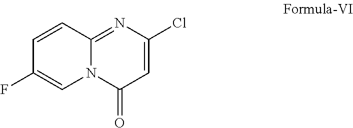 [SMILES: O=c1cc(Cl)nc2ccc(F)cn12]

in the presence of a base and catalyst in a suitable solvent to obtain compound of Formula-VII.

The present invention further provides a process for the preparation of compound of Formula-XI,

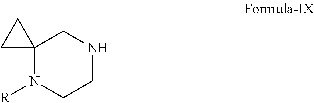

comprising the steps of:
a) reacting a compound of Formula-XII,

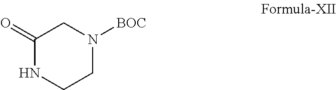 [SMILES: CC(C)(C)OC(=O)N1CCNC(=O)C1]

with benzyl bromide in presence of a base in a suitable solvent to obtain compound of Formula-XIII,

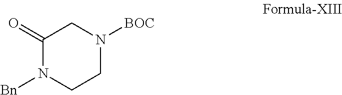 [SMILES: CC(C)(C)OC(=O)N1CCN(Cc2ccccc2)C(=O)C1]

b) treating the compound of Formula-XIII with ethyl magnesium bromide in the presence of a catalyst in suitable solvent to obtain compound of Formula-XIV,

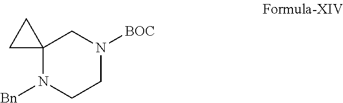 [SMILES: CC(C)(C)OC(=O)N1CCN(Cc2ccccc2)C2(CC2)C1]

c) selectively deprotecting the compound of Formula-XIV in the presence of a catalyst in a suitable solvent to obtain compound of Formula-XV,

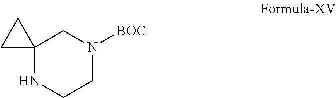 [SMILES: CC(C)(C)OC(=O)N1CCNC2(CC2)C1]

d) protecting the compound of Formula-XV in situ with amino protecting agent in the presence of a base in a suitable solvent to obtain compound of Formula-XVI,

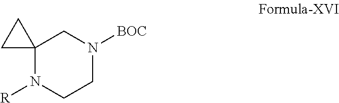

e) selectively deprotecting the compound of Formula-XVI in situ in presence of an acid in a suitable solvent to obtain compound of Formula-XI.

### DETAILED DESCRIPTION OF THE INVENTION

The present invention provides a process for the preparation of Risdiplam compound of Formula-I, comprising the steps of.
a) reacting a compound of Formula-VII with a compound of Formula-IX in presence of a base in a suitable solvent to obtain compound of Formula-X,
b) deprotecting the compound of Formula-X in presence of an acid in a suitable solvent followed by reaction with oxalic acid dihydrate to obtain Risdiplam oxalic acid salt of Formula-Ia,
c) treating the compound of Formula-Ia with an acid followed by base in a suitable solvent to obtain Risdiplam compound of Formula-I.

In step (a) of the present invention, wherein R is amine protecting group is selected from C₁-C₆ acyl, benzyloxycarbonyl (Cbz), benzyl (Bn), benzoyl (Bz), trityl, Si(C₁-C₆ alkyl)₃, mesyl, tosyl, benzenesulfonyl, triflate and thereof.

In step (a) of the present invention, wherein the base is selected from “organic bases” such as methylamine, ethylamine, diisopropylamine (DIPA), diisopropylethylamine (DIPEA), diisobutylamine, triethylamine (TEA), tert-butyl amine, pyridine, 4-dimethylaminopyridine (DMAP), N-methyl morpholine (NMM), N-methylpiperidine, 1,8-diazabicyclo[5.4.0]undec-7-ene (DBU), 1,5-diazabicyclo[4.3.0]non-5-ene (DBN), 1,4-diazabicyclo[2.2.2]octane (DABCO), imidazole and thereof.

In step (a) of the present invention, wherein the solvent is selected from polar aprotic solvents such as dimethyl sulfoxide, dimethylformamide, dimethylacetamide, sulfolane, N-methyl-2-pyrrolidone (NMP) and/or mixtures thereof.

In step (a) of the present invention, the temperature at which reaction is carried out is 120 to 130° C. for 45 to 50 hours, preferably 127.5 to 130° C. for 46 to 48 hours.

In step (a) of the present invention, compound of Formula-X is isolated from organic solvent selected from methanol, ethanol, n-propanol, isopropanol, n-butanol, isobutanol, 2-butanol, t-butanol, ethane-1,2-diol, propane-1,2-diol, toluene, xylene and/or mixture thereof.

In step (b) of the present invention, wherein the acid is selected from Lewis acid and/or inorganic acid such as BCl₃, BBr₃, AlCl₃, (C₂H₅)₂AlCl, TiCl₄ and/or Conc. HCl, HBr in acetic acid, aqueous HBr and/or mixtures thereof.

In step (b) of the present invention, wherein the solvent is selected from “chloro solvents” such as dichloromethane, dichloroethane, chloroform, carbon tetrachloride and mixture thereof and/or polar solvents such as dimethylsulphoxide, dimethylformamide, dimethylacetamide, sulfolane, N-methyl-2-pyrrolidone (NMP) and mixture thereof.

In step (b) of the present invention, the temperature at which reaction is carried out is 0 to 30° C. for 3 to 13 hours, preferably 25 to 30° C. for 12 to 13 hours.

In step (b) of the present invention, Risdiplam oxalic acid salt of Formula-Ia is isolated from organic solvent selected from dichloromethane, dichloroethane, chloroform, carbon tetrachloride, methanol, ethanol, n-propanol, isopropanol, n-butanol, isobutanol, 2-butanol, t-butanol, ethane-1,2-diol, propane-1,2-diol and/or mixture thereof.

In step (b) of the present invention, wherein the acid is selected from oxalic acid, tartartic acid, maleic acid, succinic acid and thereof.

In step (c) of the present invention, wherein the acid is selected from organic solvent such as HCl, HBr, HI, sulfuric acid, nitric acid, phosphoric acid, trifluoroacetic acid and thereof;

In step (c) of the present invention, wherein the base is selected from sodium hydroxide, potassium hydroxide, lithium hydroxide and thereof;

In step (c) of the present invention, wherein the solvent is selected from “alcohol solvents” such as methanol, ethanol, n-propanol, isopropanol, n-butanol, isobutanol, 2-butanol, t-butanol, ethane-1,2-diol, propane-1,2-diol, water and/or mixture thereof.

In step (c) of the present invention, the temperature at which reaction is carried out is 25 to 65° C. for 2 to 16 hours, preferably 60 to 65° C. for 1 to 2 hours.

In step (c) of the present invention, Risdiplam compound of Formula-I is isolated from organic solvent selected from methanol, ethanol, n-propanol, isopropanol, n-butanol, isobutanol, 2-butanol, t-butanol, ethane-1,2-diol, propane-1,2-diol, water and/or mixture thereof.

The present invention further provides a process for the preparation of compound of Formula-VII comprising the steps of:
a) reacting a compound of Formula-III with 1-bromo-2,2-dimethoxypropane in the presence of p-Toluenesulfonic acid monohydrate in a suitable solvent to obtain compound of Formula-IV;
b) treating the compound of Formula-IV with bis(pinacolato)diboron in the presence of a base and catalyst in a suitable solvent to obtain compound of Formula-V,
c) reacting the compound of Formula-V in situ with compound of Formula-VI in the presence of a base and catalyst in a suitable solvent to obtain compound of Formula-VII.

In step (a) of the present invention, wherein the solvent is selected from“alcohol solvents” such as methanol, ethanol, n-propanol, isopropanol, n-butanol, isobutanol, 2-butanol, t-butanol, ethane-1,2-diol, propane-1,2-diol and thereof.

In step (a) of the present invention, the temperature at which reaction is carried out is 70 to 80° C. for 6 to 12 hours, preferably 75 to 80° C. for 8 to 12 hours.

In step (a) of the present invention, compound of Formula-IV is isolated from organic solvent is selected from methyl acetate, ethyl acetate, n-propyl acetate, isopropyl acetate, n-butyl acetate, tert-butyl acetate, water and thereof.

In step (b) of the present invention, wherein the catalyst is selected from Pd(PPh₃)₄, PdCl₂, Pd(OAc)₂, Pd₂(dba)₃, Pd(PPh₃)₂Cl₂, PdCl₂(dppf), PdCl₂(dppf)·CH₂Cl₂, DPPF, PdCl₂(dppp), Cyclopentadienyl allyl palladium, allylpalladium(II) chloride dimer (Pd(allyl)Cl)₂, (2-Butenyl)chloropalladium dimer, (2-Methylallyl) palladium(II) chloride dimer, palladium(1-phenylallyl) chloride dimer, di-μ-chlorobis[2-(amino-N)[1,1′-biphenyl]-2-yl-C]dipalladium(II), di-μ-chlorobis[2-(dimethylamino)methyl]phenyl-C,N]dipalladium(II), dichloro [9,9-dimethyl-4,5-bis(diphenylphosphino)xanthene]palladium (Pd(XantPhos)Cl₂) and thereof.

In step (b) of the present invention, wherein the base is selected from sodium acetate, potassium acetate, lithium acetate, cesium acetate and thereof.

In step (b) of the present invention, wherein the suitable solvent is selected from acetonitrile, propionitrile, isobutyronitrile and thereof.

In step (b) of the present invention, the temperature at which reaction is carried out is 75 to 85° C. for 2 to 5 hours, preferably 75 to 80° C. for 3 to 4 hours.

The obtained compound of Formula-V can be used in the next reaction directly without isolation.

In step (c) of the present invention, wherein the catalyst is selected from Pd(PPh₃)₄, PdCl₂, Pd(OAc)₂,Pd₂(dba)₃, Pd(PPh₃)₂Cl₂, PdCl₂(dppf), PdCl₂ (dppf)·CH₂Cl₂, PdCl₂(dppp), Cyclopentadienyl allyl palladium, allylpalladium(II) chloride dimer (Pd(allyl)Cl)₂, (2-Butenyl)chloropalladium dimer, (2-Methylallyl) palladium(II) chloride dimer, palladium(1-phenylallyl)chloride dimer, di-μ-chlorobis[2-(amino-N)[1,1′-biphenyl]-2-yl-C]dipalladium(II), di-μ-chlorobis[2-(dimethylamino)methyl]phenyl-C,N]dipalladium(II), dichloro[9,9-dimethyl-4,5-bis(diphenylphosphino)xanthene]palladium (Pd(XantPhos)Cl₂) and thereof.

In step (c) of the present invention, wherein the base is selected from sodium carbonate, potassium carbonate, lithium carbonate, cesium carbonate and thereof.

In step (c) of the present invention, wherein the suitable selected from acetonitrile, propionitrile, isobutyronitrile and thereof.

In step (c) of the present invention, the temperature at which reaction is carried out is 70 to 80° C. for 3 to 7 hours, preferably 75 to 80° C. for 3 to 4 hours.

The obtained compound of Formula-VII is isolated from the solvent selected from methanol, ethanol, n-propanol, isopropanol, n-butanol, isobutanol, 2-butanol, t-butanol, ethane-1,2-diol, propane-1,2-diol and thereof.

The present invention further provides a process for the preparation of compound of Formula-XI comprising the steps of:
a) reacting a compound of Formula-XII with benzyl bromide in the presence of a base in a suitable solvent to obtain compound of Formula-XIII,
b) treating the compound of Formula-XIII with ethyl magnesium bromide in the presence of a catalyst in a suitable solvent to obtain compound of Formula-XIV,
c) selectively deprotecting the compound of Formula-XIV in the presence of a catalyst in a suitable solvent to obtain compound of Formula-XV,
d) protecting the compound of Formula-XV in situ with amino protecting agent in the presence of a base in a suitable solvent to obtain compound of Formula-XVI,
e) selectively deprotecting the compound of Formula-XVI in situ in the presence of an acid in a suitable solvent to obtain compound of Formula-XI.

In step (a) of the present invention, wherein the base is selected from lithium hydride, sodium hydride, potassium-hydride, rubidium hydride, cesium hydride and thereof.

In step (a) of the present invention, wherein the solvent is selected from dimethyl ether, diisopropyl ether, diethyl ether, methyl tert-butyl ether, 1,2-dimethoxyethane, tetrahydrofuran, 1,4-dioxane, monoglyme, diglyme and/or mixture thereof.

In step (a) of the present invention, the temperature at which reaction is carried out is 10 to 30° C. for 1 to 12 hours, preferably 25 to 30° C. for 10 to 12 hours.

In step (a) of the present invention, compound of Formula-XIII is isolated from organic solvent selected from n-hexane, n-heptane, cyclohexane, petroleum ether and thereof.

In step (b) of the present invention, wherein the catalyst is selected from ClTi(OiPr)₃, Ti(OiPr)₄, ClTi(OtBu)₃, Ti(OtBu)₄ and thereof.

In step (b) of the present invention, wherein the solvent is selected from dimethyl ether, diisopropyl ether, diethyl ether, methyl tert-butyl ether, 1,2-dimethoxyethane, tetrahydrofuran, 1,4-dioxane, monoglyme, diglyme and/or mixtures thereof.

In step (b) of the present invention, the temperature at which reaction is carried out is 60 to 65° C. for 1 to 1.5 hours and 25 to 30° C. for 10 to 12 hours.

In step (c) of the present invention, wherein the catalyst is selected from Pd(OH)₂/C, Pt, Pt/C, PtO₂, Pd, Pd/C, Rh, Ru, Ni or Raney-Ni, Zn, Sn or Fe and an acid; AlH₃—AlCl₃; hydrazine and a catalyst; [Fe₃(CO)₁₂]-methanol; hot liquid paraffin; formic acid or ammonium formate, Pd/C; LiAlH₄, NaHS, (NH₄)₂S or polysulfides and thereof.

In step (c) of the present invention, wherein the solvent is selected from methanol, ethanol, n-propanol, isopropanol, n-butanol, isobutanol, 2-butanol, t-butanol, ethane-1,2-diol, propane-1,2-diol and thereof.

In step (c) of the present invention, the temperature at which reaction is carried out is 25 to 30° C. for 5 to 10 hours, preferably for 5 to 6 hours. The obtained compound of Formula-XV can be used in the next reaction directly without isolation.

In step (d) of the present invention, wherein the amino protecting agent is selected from C₁-C₆ acyl, benzyloxycarbonyl (Cbz), benzyl (Bn), benzoyl (Bz), trityl, Si(C₁-C₆ alkyl)₃, mesyl, tosyl, benzenesulfonyl, triflate and thereof.

In step (d) of the present invention, wherein the base is selected from “organic bases” such as methylamine, ethylamine, diisopropylamine (DIPA), diisopropylethylamine (DIPEA), diisobutylamine, triethylamine (TEA), tert-butyl amine, pyridine, 4-dimethylaminopyridine (DMAP), N-methyl morpholine (NMM), N-methylpiperidine, 1,8-diazabicyclo[5.4.0]undec-7-ene (DBU), 1,5-diazabicyclo[4.3.0]non-5-ene (DBN), 1,4-diazabicyclo[2.2.2]octane (DABCO), imidazole and thereof.

In step (d) of the present invention, wherein the solvent is selected from “chloro solvents” such as dichloromethane, dichloroethane, chloroform, carbon tetrachloride and/or mixture thereof.

In step (d) of the present invention, the temperature at which reaction is carried out is 0 to 30° C. for 1 to 2 hours, preferably 25 to 30° C. for 1 to 1.5 hours.

The obtained compound of Formula-XVI can be used in the next reaction directly without isolation In step (e) of the present invention, wherein the acid is selected from HCl, HBr, HI, and thereof.

In step (e) of the present invention, wherein the solvent is selected from methyl acetate, ethyl acetate, n-propyl acetate, isopropyl acetate, n-butyl acetate, tert-butyl acetate and thereof.

In step (e) of the present invention, the temperature at which reaction is carried out is 25 to 30° C. for 6 to 12 hours, preferably for 8 to 10 hours.

In step (d) of the present invention, the reaction mixture further treated with an acid such as tartartic acid, maleic acid, succinic acid and thereof; followed by treating with base such as sodium hydroxide, potassium hydroxide, lithium hydroxide and thereof to obtain compound of Formula-XI,

### Advantages of Present Invention

1. Novel process was developed for the compound of Formula-XIa.
2. A telescoped process was developed for the compound of Formula-VIII from compound of Formula-XIV without isolating compound of Formula-XV and XVIa.
3. Novel intermediates were prepared with good purity.
4. Risdiplam was isolated as pure crystalline powder with >99.85% purity.

Scheme-II: A process for the preparation of Risdiplam compound of Formula-I, which is shown below:

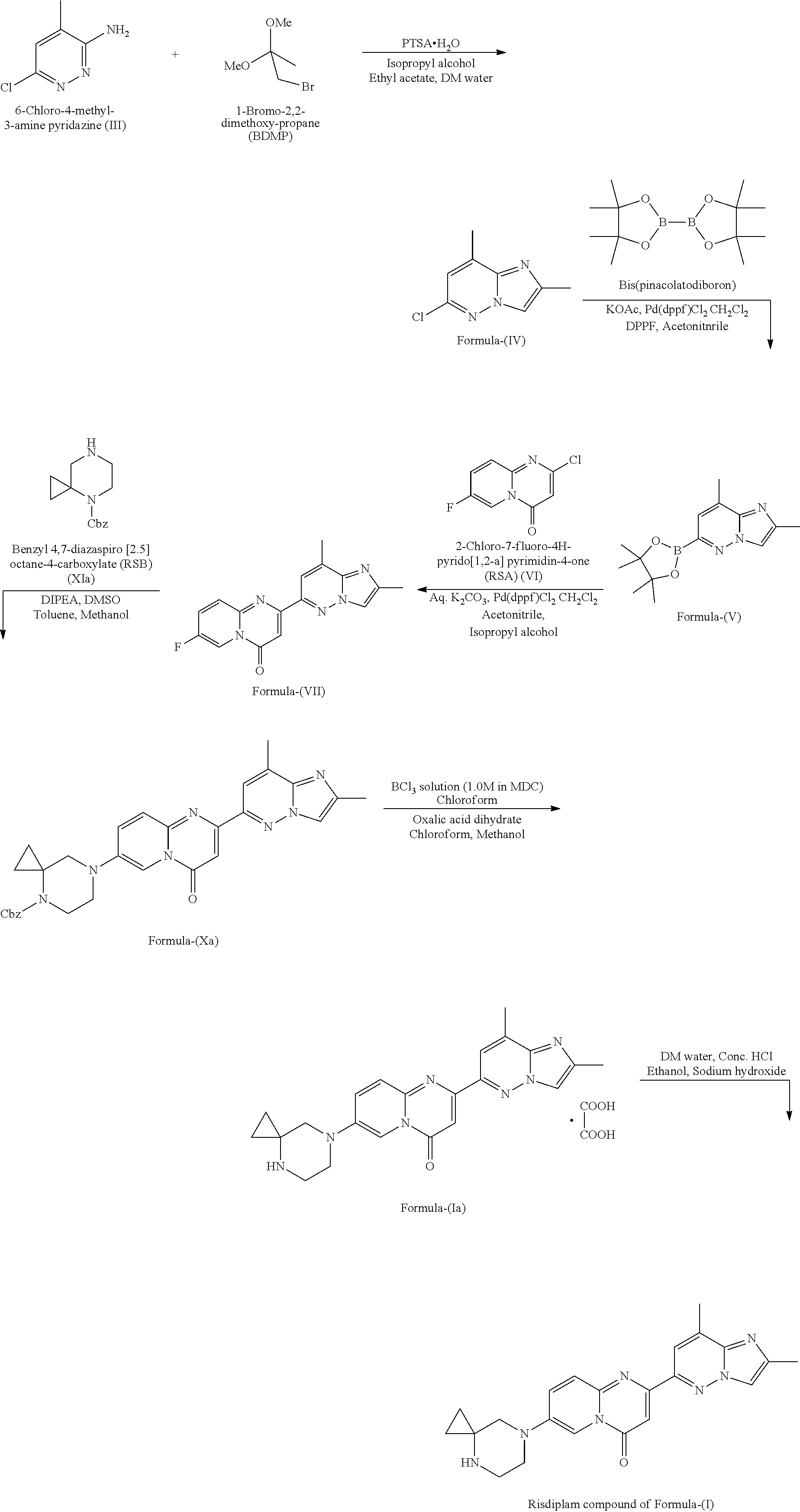

Scheme-III: A process for the preparation of intermediate compound of Formula-VI, which is shown below:

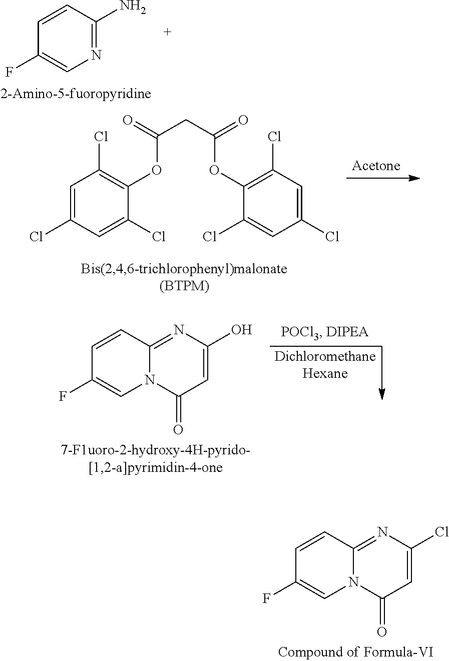 [SMILES: Nc1ccc(F)cn1 ; O=C(CC(=O)Oc1c(Cl)cc(Cl)cc1Cl)Oc1c(Cl)cc(Cl)cc1Cl ; O=c1cc(O)nc2ccc(F)cn12 ; O=c1cc(Cl)nc2ccc(F)cn12]

Scheme-IV: A process for the preparation of intermediate compound of Formula-XIa, which is shown below:

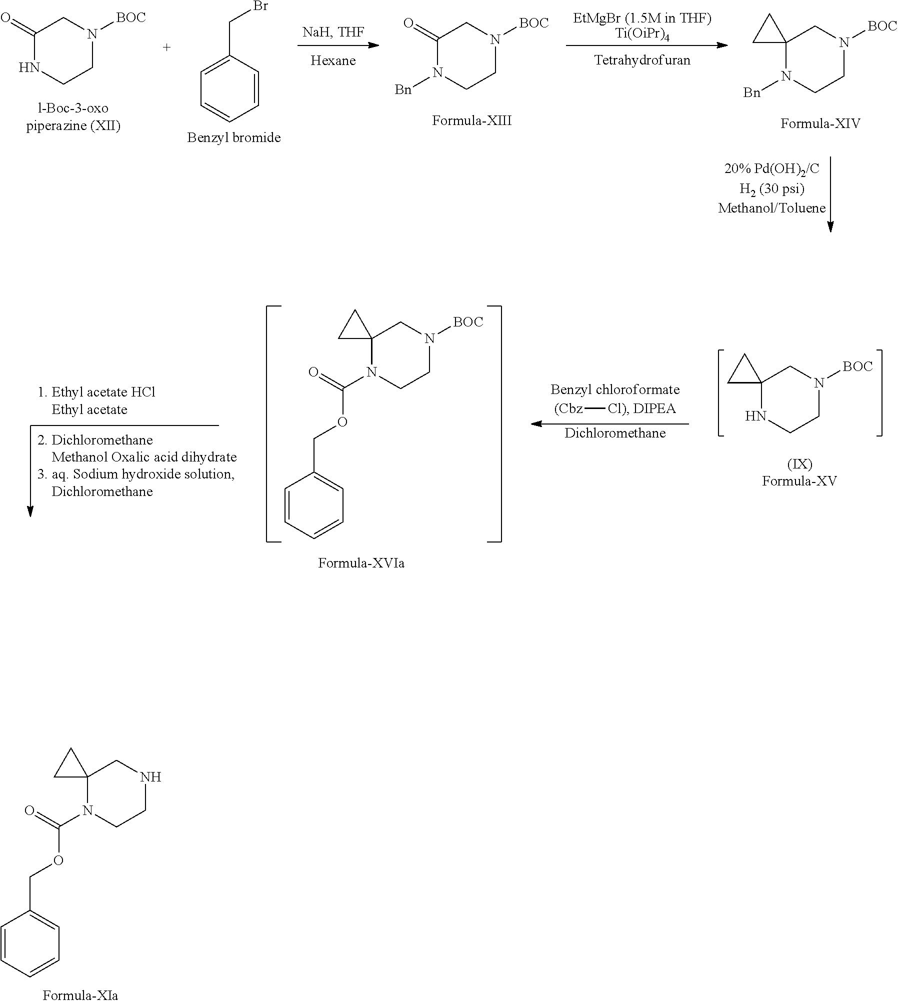

The following examples are provided to illustrate the invention and are merely for illustrative purpose only and should not be construed to limit the scope of the invention.

### EXAMPLES

### Example-1: Preparation of 6-Chloro-2,8-dimethylimidazo[1,2-b]pyridazine (Formula-IV)

To a stirred suspension of 6-chloro-4-methyl-3-aminopyridazine (Formula-III) (200 g 1.0 equiv.) in Isopropyl alcohol (1.2 L), 1-bromo-2,2-dimethoxypropane (305.97 g, 1.2 equiv.) was added and stirred for 10-20 min at 27.5±2.5° C. p-Toluenesulfonic acid monohydrate Lot-I (26.49 g, 0.1 equiv.) was added to the reaction mass, heated to 77.5±2.5° C. and stirred for 3.0 hours. 1-Bromo-2,2-dimethoxypropane (127.49 g, 0.5 equiv.) and p-Toluenesulfonic acid monohydrate (26.49 g, 0.1 equiv.) were added again to the reaction mass and stirred for another 3.0 hours at 77.5±2.5° C. After completion of the reaction (by HPLC), reaction mass was cooled to 27.5±2.5° C. Then DM water (4 L) was added to the reaction mass, pH was adjusted to 9.0-10.0 with aq. sodium hydroxide solution and stirred for 3.0 hours at 27.5±2.5° C. Thereafter, reaction mass was further cooled to 2.5±2.5° C., stirred for 3.0 hours, filtered and washed with DM water (1 L). A mixture of wet solid, ethyl acetate (6 L) and DM water (1 L) were stirred for 10-15 min, filtered the mass through hyflo bed and washed with ethyl acetate (1 L). Both layers were separated and the organic layer was treated with activated carbon (20 g) for 45-60 min at 27.5±2.5° C. Filtered the mass through hyflo bed and washed with ethyl acetate (1 L). Filtrate was concentrated under reduced pressure at below 45° C. to get crude material of the title compound. Crude material was leached with DM water (4 L) for 1.0-1.5 hours at 27.5±2.5° C., filtered, washed with DM water (1 L) and dried under vacuum for 14.0 hours at 40±2.5° C. to get 6-chloro-2,8-dimethylimidazo[1,2-b]pyridazine of Formula-IV as an off-white solid.

Yield: 172.0 g (67.98%); Purity: 99.86%

### Example-2: Preparation of 2-(2,8-Dimethylimidazo[1,2-b]pyridazin-6-yl)-7-fluoro-4H-pyrido[1,2-a]pyrimidin-4-one (Formula-VII)

Evacuated the RBF with argon gas. Charged Acetonitrile (1.75 L) into RBF and purged with argon for 30-45 min at 27.5±2.5° C. To this added, 6-chloro-2, 8-dimethylimidazo[1,2-b]pyridazine (Formula-IV) (125 g, 1.0 equiv.), bis(pinacolato)diboron (218.11 g, 1.25 equiv.) and potassium acetate (135.09 g, 2.0 equiv.) and purged with argon for 45 min at 27.5±2.5° C. 1,1′-bis(diphenylphosphino)ferrocene]dichloropalladium(II), complex with dichloromethane [Pd(dppf)Cl₂. MDC complex], 11.24 g, 0.02 equiv.) and 1,1′-bis(diphenylphosphino)ferrocene (DPPF, 1.91 g, 0.005 equiv.) were added to the reaction mass and purged with argon for 30 min at 27.5±2.5° C. The reaction mass was heated to 80±2.5° C. and stirred for 4.0 hours. After completion of the reaction (by HPLC), reaction mass was cooled to 27.5±2.5° C., stirred for 1.5 hour, filtered and washed with acetonitrile (3×250 mL) to get 2,8-dimethyl-6-(4,4,5,5-tetramethyl-1,3,2-dioxaborolan-2-yl)imidazo[1,2-b]pyridazine (Formula-V) in acetonitrile solution. 2-Chloro-7-fluoro-4H-pyrido[1,2-a]pyrimidin-4-one (Formula-VI) (116.16 g, 0.85 equiv.) and aq. potassium carbonate solution [190.23 g of potassium carbonate was dissolved in 750 mL of DM water] were added to the above acetonitrile solution and purged with argon for 45-60 min. 1,1′-Bis(diphenylphosphino)ferrocene]dichloropalladium(II), complex with dichloromethane [Pd(dppf)Cl₂. MDC complex, 11.24 g, 0.02 equiv.] was added to the reaction mass, heated to 77.5±2.5° C. and stirred for 4.0 hours. After completion of the reaction (by HPLC), reaction mass was cooled to 27.5±2.5° C. and stirred for 1.5 hour. The precipitated solid was filtered and washed with acetonitrile (2×250 mL). The wet solid was leached with DM water (1.25 L) for 1.5 hour at 52.5±2.5° C., filtered and washed with DM water (625 mL). Thereafter, wet solid was further leached with Isopropyl alcohol (625 mL) for 1.5 hour at 27.5±2.5° C., filtered, washed with Isopropyl alcohol (2×187.5 mL) and dried under vacuum for 12.0 hours at 67.5±2.5° C. to get title compound as dark green coloured solid.

Yield: 155.2 g (72.91%); Purity: 97.59%

### Example-3: Preparation of Benzyl 7-[2-(2,8-dimethylimidazo[1,2-b]pyridazin-6-yl)-4-oxo-pyrido[1,2-a]pyrimidin-7-yl]-4,7-diazaspiro[2.5]octane-4-carboxylate (Formula-Xa)

To a stirred suspension of 2-(2,8-dimethylimidazo[1,2-b]pyridazin-6-yl)-7-fluoro-4H-pyrido[1,2-a]pyrimidin-4-one (Formula-VII) (20 g, 1.0 equiv.), benzyl 4,7-diazaspiro[2.5]octane-4-carboxylate (Formula-XIa) (27.87 g, 1.75 equiv.) in DMSO (160 mL), DIPEA (50.13 g, 6.0 equiv.) was added, heated to 127.5±2.5° C. and stirred for 48 hours. After completion of the reaction (by HPLC), reaction mass was cooled to 27.5±2.5° C. and stirred for 45 min. Isopropyl alcohol (160 mL) was added to the reaction mass and stirred for 60 min at 27.5±2.5° C. Thereafter, DM water (80 mL) was added to the reaction mass and stirred for 2.0 h at 27.5±2.5° C., filtered and washed with DM water (2×100 mL). The wet solid was dissolved in Chloroform (500 mL) and treated with aq. N-Acetyl-L-cysteine solution (100 mL), filtered through hyflo bed and separated the both layers. Organic layer was again treated with aq. N-Acetyl-L-cysteine solution (30 mL) and separated the both layers. Thereafter, org. layer was treated with 50% aq. HCl solution (20 mL) for 60 min at 27.5±2.5° C., filtered and washed with chloroform (60 mL). Wet solid was dissolved in DM water (200 mL), adjusted the reaction mass pH to 10-11 with aq. sodium hydroxide solution at 27.5±2.5° C., filtered and washed with DM water (100 mL). A mixture methanol (200 mL), toluene (40 mL) and above wet solid were heated and stirred for 1.5 h at 62.5±2.5° C. Later, reaction mass was cooled to 27.5±2.5° C., stirred for 3.0 h, filtered, washed with methanol (60 mL) and dried the material under vacuum for 8.0 h at 67.5±2.5° C. to get title compound.

Yield: 16.4 g (47.35%); Purity: 99.22%

### Example-4: Preparation of 7-(4,7-Diazaspiro[2.5]oct-7-yl)-2-(2,8-dimethylimidazo[1,2-b]pyridazin-6-yl)-4H-pyrido[1,2-a]pyrimidin-4-one, oxalic acid (1:1) (Formula-Ia)

To a stirred suspension of benzyl 7-[2-(2,8-dimethylimidazo[1,2-b]pyridazin-6-yl)-4-oxo-pyrido[1,2-a]pyrimidin-7-yl]-4,7-diazaspiro[2.5]octane-4-carboxylate (Formula-Xa) (70 g, 1.0 equiv.) in chloroform (1400 mL), boron trichloride solution (BCl₃, 1.0M in MDC, 653.5 mL, 5.0 equiv.) was added at below −10° C., then stirred at 2.5±2.5° C. for 4.0 h followed by 13.0 h at 27.5±2.5° C. affords Formula-Ia as a free base. This was further treated with L-(+)-tartaric acid followed by maleic acid. Later Formula-Ia free base (33.79 g, 1.0 equiv.) was treated with oxalic acid dihydrate (10.61 g, 1.0 equiv.) in chloroform and methanol solvent mixture for 2.0 h at 27.5±2.5° C., filtered, washed with methanol and dried under vacuum for 10.0 h at 62.5±2.5° C. to get title compound as yellow color powder.

Yield: 38.3 g (59.63%); Purity: 99.73%

### Example-5: Preparation of 7-(4,7-Diazaspiro[2.5]oct-7-yl)-2-(2,8-dimethylimidazo[1,2-b]pyridazin-6-yl)-4H-pyrido [1,2-a]pyrimidin-4-one (Formula-I)

A solution of 7-(4,7-diazaspiro[2.5]oct-7-yl)-2-(2,8-dimethylimidazo[1,2-b]pyridazin-6-yl)-4H-pyrido[1,2-a]pyrimidin-4-one, oxalic acid (1:1) (Formula-Ia) (28 g, 1.0 equiv.) and aq. HCl (11.21 g, 2.0 equiv.) was added into sodium hydroxide (11.38 g, 5.0 equiv.) in ethanol solution at 62.5±2.5° C. and stirred for 2.0 h. There after, reaction mass was cooled to 27.5±2.5° C., stirred for 16.0 h, filtered and washed with ethanol (56 mL). The wet solid was leached with DM water (280 mL) for 1.5 h at 27.5±2.5° C., filtered, washed with DM water and dried the material under vacuum for 12.0 h at 57.5±2.5° C. to get title compound as yellow colour crystalline powder.

Yield: 18.2 g (79.58%); Purity: 99.85%

### Example-6: Preparation of 2-Chloro-7-fluoro-4H-pyrido[1,2-a]pyrimidin-4-one (Formula-VI)

### Step-I:

To a stirred solution of 2-amino-5-fluoropyridine (40 g 1.0 equiv.) in acetone (1.6 L), bis(2,4,6-trichlorophenyl)malonate (198.12 g, 1.2 equiv.) was added and stirred for 24.0 hours at 27.5±2.5° C. After completion of the reaction (by TLC), filtered the solid and washed with acetone (80 mL). The wet solid was leached with acetone (200 mL) for 1.5 hour at 27.5±2.5° C., filtered, washed with acetone (80 mL) and dried under vacuum for 7.0 hours at 55-60° C. to get 7-Fluoro-2-hydroxy-4H-pyrido[1,2-a]pyrimidin-4-one as yellow coloured powder.

Yield: 57.90 g (90.08%); Purity: 97.45%

### Step-II:

To a stirred suspension of 7-fluoro-2-hydroxy-4H-pyrido [1,2-a]pyrimidin-4-one (45 g 1.0 equiv.) in phosphorous oxychloride (268.1 g, 7.0 equiv.), DIPEA (32.28 g, 1.0 equiv.) was added at 30±5° C. Reaction mass temperature was raised to 105±2.5° C. and stirred for 7.0 h. After completion of the reaction (by HPLC), reaction mass was concentrated under reduced pressure at <120° C. Then reaction mass was cooled to 7.5±2.5° C., pre-cooled DM water (675.0 mL) was added and stirred for 15 min. Thereafter, reaction mass temperature was raised to 27.5±2.5° C., stirred for 2.0 hours, filtered and washed with DM water (90 mL). The wet solid was dissolved in dichloromethane (1.575 L) and washed with 5% aq. sodium carbonate solution (112.5 mL) followed by DM water (112.5 mL). Both layers were separated and the organic layer treated with activated carbon (4.5 g) for 1.5 hour at 27.5±2.5° C., filtered through hyflo and washed with dichloromethane (135 mL). Organic layer was concentrated under reduced pressure at <40° C. and co-distilled with hexanes (90 mL) to get residue. A mixture of above residue and hexanes (225 mL) were stirred for 1.5 hour at 27.5±2.5° C., filtered, washed with hexanes (45 mL) and dried under vacuum for 6.0 hours at 57.5±2.5° C. to get title compound as yellow coloured powder.

Yield: 41.0 g (82.66%); Purity: 99.35%

### Example-7: Preparation of tert-Butyl 4-benzyl-3-oxo-piperazine-1-carboxylate (Formula-XIII)

To a pre-cooled suspension of tert-butyl 3-oxopiperazine-1-carboxylate (Formula-XII) (400 g, 1.0 equiv.) in THF (2.8 L), NaH (60%, 103.88 g, 1.3 equiv.) was added lot wise at 0±5° C. and stirred for 2.0 h at 10±2.5° C. Benzyl bromide (375.83 g, 1.1 equiv.) was added to the reaction mass at 0±5° C., reaction mass temperature was raised to 27.5±2.5° C. and stirred for 12.0 hours. After completion of the reaction (by TLC), quenched the reaction mass with 10% aqueous ammonium chloride solution (2 L) and extracted twice with ethyl acetate (1.6 L and 0.8 L). Organic layer washed twice with aq. sodium bicarbonate solution (2×2 L) followed by aq. sodium chloride solution (2 L). Organic layer was concentrated under reduced pressure and co-distilled with hexanes (1.2 L) at <55° C. to get crude compound. The crude compound was leached with hexanes (2 L) for 1.5 h at 62.5±2.5° C., then stirred the mass for 1.5 h at 27.5±2.5° C. filtered, washed with hexanes (3×1.2 L) and dried in hot air oven for 12.0 h at 52.5±2.5° C. to get title compound as an off-white coloured solid.

Yield: 517 g (89.13%); Purity: 99.65%

### Example-8: tert-Butyl 4-benzyl-4,7-diazaspiro[2.5]octane-7-carboxylate (Formula-XIV)

To a pre-cooled solution of tert-butyl 4-benzyl-3-oxo-piperazine-1-carboxylate (Formula-XIII) (25 g, 1.0 equiv.) and Titanium(IV) isopropoxide (29.36 g, 1.2 equiv.) in THF (250 mL), Ethylmagnesium bromide (147.6 mL, 1.5M in THF, 3.0 equiv.) was added at 20±2.5° C. under nitrogen atmosphere and stirred the reaction mass for 9.0-10.0 h at 27.5±2.5° C. Titanium(IV) isopropoxide (14.68 g, 0.6 equiv.) and Ethylmagnesium bromide (73.77 mL, 1.5M in THF, 1.5 equiv.) were added again to the reaction mass at 20±2.5° C. under nitrogen atmosphere and stirred the reaction mass for 6.0 h at 27.5±2.5° C. After completion of the reaction (by HPLC), quenched the reaction mass with aq. ammonium chloride solution (375 mL), filtered through Hyflo bed and extracted with ethyl acetate (400 mL). Organic layer was washed with aq. sodium chloride solution and concentrated under reduced pressure at <55° C. and resulting crude compound was purified by silica gel column chromatography to get title compound as light yellow color viscous liquid.

Yield: 15 g (57.62%); Purity: 94.50%

### Example-9: Preparation of tert-Butyl 4,7-diazaspiro[2.5]octane-7-carboxylate (Formula-XV)

To a solution of tert-butyl 4-benzyl-4,7-diazaspiro[2.5]octane-7-carboxylate (Formula-XIV) (50 g, 1.0 equiv.) in methanol (500 mL), 20% Pd(OH)₂/C (50% wet) (3.5 g, 7% w/w) was added and the reaction mass was stirred for 6.0 hours in an autoclave vessel at 27.5±2.5° C. by using hydrogen gas (30 psi). After completion of the reaction (by TLC), filtered the reaction mass through Hyflo bed, washed with methanol (150 mL) and filtrate was concentrated and co-distilled with toluene (150 mL) under reduced pressure at <55° C. to get title compound as pale brown coloured oily mass.

Yield: 33.8 g (96.29%)

### Example-10: Preparation of 4-benzyl-7-tert-butyl-4,7-diazaspiro[2.5]octane-4,7-dicarboxylate (Formula-XVIa)

To a stirred solution of tert-butyl 4,7-diazaspiro[2.5]octane-7-carboxylate (Formula-XV) (33 g, 1.0 equiv.) in dichloromethane (198 mL), DIPEA (30.13 g, 1.5 equiv.) was added at 27.5±2.5° C. Reaction mass was cooled to 0-5° C. and added benzyl chloroformate (50% in Toluene, 53.02 g, 1.0 equiv.) at 0-5° C. Then the reaction mass temperature was raised to 27.5±2.5° C. and stirred for 1.5 hour. After completion of the reaction (by TLC), aq. HCl solution (264 mL) was added to reaction mass and stirred for 10-15 min. Both layers were separated, and the organic layer was washed with aq. HCl solution (264 mL) followed by DM water (264 mL) and concentrated under reduced pressure at <50° C. to get title compound as residue.

Yield: 56.2 g (Quantitative)

### Example-11: Preparation of Benzyl 4,7-diazaspiro[2.5]octane-4-carboxylate (Formula-XIa)

To a stirred solution of 4-benzyl-7-tert-butyl-4,7-diazaspiro[2.5]octane-4,7-dicarboxylate (Formula-XVIa) (53.84 g, 1.0 equiv.) in ethyl acetate (270 mL), HCl in ethyl acetate (16.3% w/w, 173.8 g, 5.0 equiv.) was added at 10-15° C. and stirred for 10.0 h at 27.5±2.5° C. After completion of the reaction (by TLC), quenched the reaction mass with DM Water (162 mL) and separated both the layers. Aq. layer was washed with MTBE (270 mL) and diluted with aq. sodium hydroxide solution (243 mL). Product was extracted with dichloromethane (4×162 mL) and oxalic acid dihydrate solution in Methanol (21.56 g, 1.1 equiv.) was added to dichloromethane layer, stirred for 4.0 h at 27.5±2.5° C., filtered and washed with dichloromethane (162 mL). The obtained product was basified with aq. sodium hydroxide solution (1350 mL) and extracted with dichloromethane (3×270 mL) and combined organic layer was washed with aq. sodium chloride solution (108 mL). Organic layer dried over anhydrous sodium sulphate (54 g) and concentrated under reduced pressure at ≤40° C. to get title compound as off-white to yellow or brown colour viscous liquid (solidifies on standing).

Yield: 28.3 g (73.94%); Purity: 97.87%

We claim:

1. A process for the preparation of Risdiplam compound of Formula-I,
comprising the steps of: 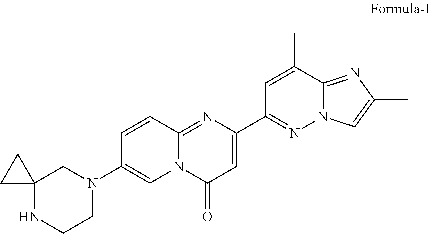 [SMILES: Cc1cn2nc(-c3cc(=O)n4cc(N5CCNC6(CC6)C5)ccc4n3)cc(C)c2n1]
a) reacting a compound of Formula-VII, 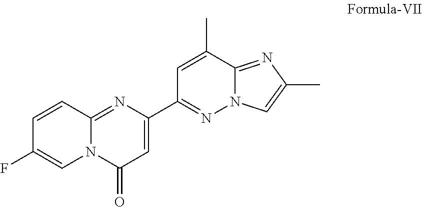 [SMILES: Cc1cn2nc(-c3cc(=O)n4cc(F)ccc4n3)cc(C)c2n1]
with a compound of Formula-IX, 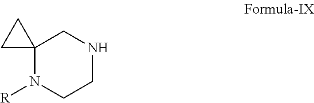
wherein, R is amine protecting group other than Boc (tert-butoxy carbonyl);
in presence of a base in a suitable solvent to obtain compound of Formula-X, 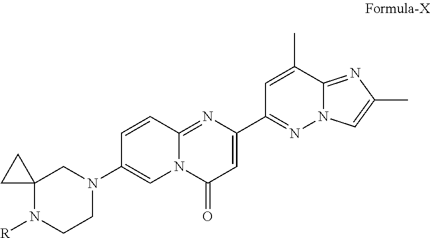
b) deprotecting the compound of Formula-X in presence of an acid in a suitable solvent followed by reaction with oxalic acid dihydrate to obtain Risdiplam oxalic acid salt of Formula-Ia. 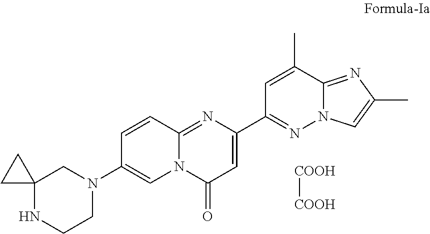
c) treating the compound of Formula-Ia with an acid followed by base in a suitable solvent to obtain Risdiplam compound of Formula-I.

2. The process as claimed in claim 1, wherein,
In step-a) “R” is amino protecting group is selected from C₁-C₆ acyl, benzyloxycarbonyl (Cbz), benzyl (Bn), benzoyl (Bz), trityl, Si(C₁-C₆ alkyl)₃, mesyl, tosyl, benzenesulfonyl, triflate and thereof. The base used is selected from “organic bases” such as methylamine, ethylamine, diisopropylamine (DIPA), diisopropylethylamine (DIPEA), diisobutylamine, triethylamine (TEA), tert-butyl amine, pyridine, 4-dimethylaminopyridine (DMAP), N-methyl morpholine (NMM), N-methylpiperidine, 1,8-diazabicyclo [5.4.0]undec-7-ene (DBU), 1,5-diazabicyclo[4.3.0]non-5-ene (DBN), 1,4-diazabicyclo[2.2.2]octane (DABCO), imidazole and thereof. The suitable solvent used is selected from polar aprotic solvents such as dimethyl sulfoxide, dimethylformamide, dimethylacetamide, sulfolane, N-methyl-2-pyrrolidone (NMP) and/or mixtures thereof;
In step-b) the acid used is selected from Lewis acid and/or inorganic acid such as BCl₃, BBr₃, AlCl₃, (C₂H₅)₂AlCl, TiCl₄ and/or Conc. HCl, HBr in acetic acid, aqueous HBr and/or mixtures thereof. The suitable solvent used is selected from “chloro solvents” such as dichloromethane, dichloroethane, chloroform, carbon tetrachloride and mixture thereof and/or polar solvents such as dimethylsulphoxide, dimethylformamide, dimethylacetamide, sulfolane, N-methyl-2-pyrrolidone (NMP) or mixture thereof;
In step-c) the acid used is selected from HCl, HBr, HI, sulfuric acid, nitric acid, phosphoric acid, trifluoroacetic acid and thereof; the base used is selected from sodium hydroxide, potassium hydroxide, lithium hydroxide and thereof. The solvent used is selected from “alcohol solvents” such as methanol, ethanol, n-propanol, isopropanol, n-butanol, isobutanol, 2-butanol, t-butanol, ethane-1,2-diol, propane-1,2-diol, water and/or mixture thereof.

3. A process for the preparation of Risdiplam compound of Formula-I, comprising the steps of:
a) reacting a compound of Formula-VII, 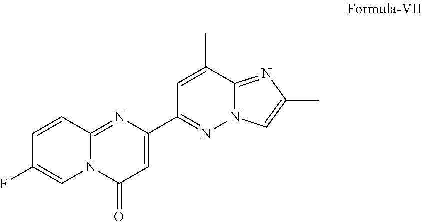 [SMILES: Cc1cn2nc(-c3cc(=O)n4cc(F)ccc4n3)cc(C)c2n1]
with a compound Formula-IXa, 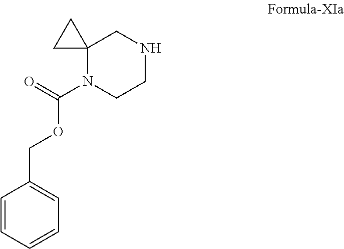 [SMILES: O=C(OCc1ccccc1)N1CCNCC12CC2]
in presence of a base in a suitable solvent to obtain compound of Formula-Xa, 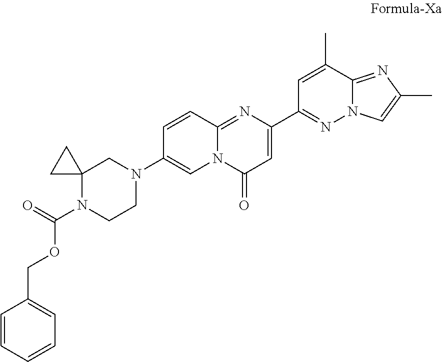 [SMILES: Cc1cn2nc(-c3cc(=O)n4cc(N5CCN(C(=O)OCc6ccccc6)C6(CC6)C5)ccc4n3)cc(C)c2n1]
b) deprotecting the compound of Formula-Xa in presence of an acid in a suitable solvent followed by reaction with oxalic acid dihydrate to obtain Risdiplam oxalic acid salt of Formula-Ia. 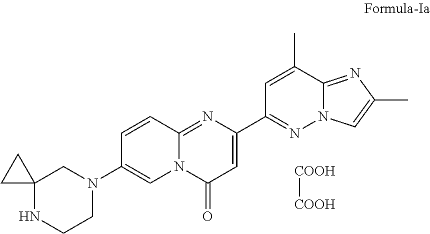
c) treating the compound of Formula-Ia with an acid followed by base in a suitable solvent to obtain Risdiplam compound of Formula-I.

4. The process as claimed in claim 3, wherein,
In step-a) the base used is selected from “organic bases” such as methylamine, ethylamine, diisopropylamine (DIPA), diisopropylethylamine (DIPEA), diisobutylamine, triethylamine (TEA), tert-butyl amine, pyridine, 4-dimethyl aminopyridine (DMAP), N-methyl morpholine (NMM), N-methylpiperidine, 1,8-diazabicyclo[5.4.0]undec-7-ene (DBU), 1,5-diazabicyclo[4.3.0]non-5-ene (DBN), 1,4-diazabicyclo[2.2.2]octane (DABCO), imidazole and thereof. The suitable solvent used is selected from polar aprotic solvents such as dimethyl sulfoxide, dimethylformamide, dimethylacetamide, sulfolane, N-methyl-2-pyrrolidone (NMP) and/or mixtures thereof;
In step-b) the acid used is selected from Lewis acid and/or inorganic acid such as BCl₃, BBr₃, AlCl₃, (C₂H₅)₂AlCl, TiCl₄ and/or Conc. HCl, HBr in acetic acid, aqueous HBr and/or mixtures thereof. The suitable solvent used is selected from “chloro solvents” such as dichloromethane, dichloroethane, chloroform, carbon tetrachloride and mixture thereof and/or polar solvents such as dimethylsulphoxide, dimethylformamide, dimethylacetamide, sulfolane, N-methyl-2-pyrrolidone (NMP) and mixture thereof;
In step-c) the acid used is selected from HCl, HBr, HI, sulfuric acid, nitric acid, phosphoric acid, trifluoroacetic acid and thereof; the base used is selected from sodium hydroxide, potassium hydroxide, lithium hydroxide and thereof. The solvent used is selected from “alcohol solvents” such as methanol, ethanol, n-propanol, isopropanol, n-butanol, isobutanol, 2-butanol, t-butanol, ethane-1,2-diol, propane-1,2-diol, water and/or mixture thereof.

5. A process for the preparation of compound of Formula-VII, 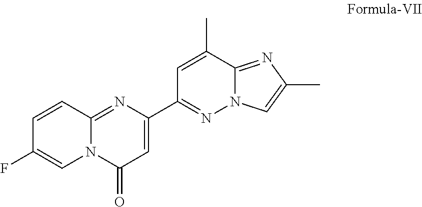 [SMILES: Cc1cn2nc(-c3cc(=O)n4cc(F)ccc4n3)cc(C)c2n1]
comprising the steps of:
a) reacting a compound of Formula-III 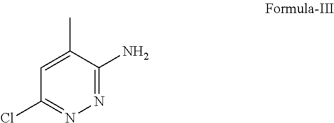
with 1-bromo-2,2-dimethoxypropane in presence of p-Toluenesulfonic acid monohydrate in a suitable solvent to obtain compound of Formula-IV 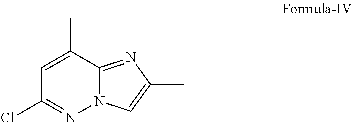
b) treating the compound of Formula-IV with bis(pinacolato)diboron in the presence of a base and catalyst in a suitable solvent to obtain compound of Formula-V; 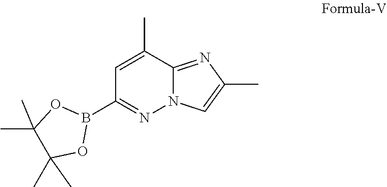
c) reacting the compound of Formula-V in situ with compound of Formula-VI 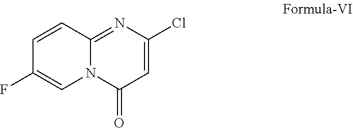 [SMILES: O=c1cc(Cl)nc2ccc(F)cn12]
in the presence of a base and a catalyst in a suitable solvent to obtain compound of Formula-VII.

6. The process as claimed in claim 5, wherein,
In step-a) the suitable solvent used is selected from organic solvent such as methyl acetate, ethyl acetate, n-propyl acetate, isopropyl acetate, n-butyl acetate, tert-butyl acetate; and water or mixture thereof;
In step-b) the catalyst used is selected from Pd(PPh₃)₄, PdCl₂, Pd(OAc)₂,Pd₂(dba)₃, Pd(PPh₃)₂Cl₂, PdCl₂(dppf), PdCl₂(dppf)·CH₂Cl₂, DPPF, PdCl₂(dppp), Cyclopentadienyl allyl palladium, allylpalladium(II) chloride dimer (Pd(allyl)Cl)₂, (2-Butenyl)chloropalladium dimer, (2-Methylallyl) palladium(II) chloride dimer, palladium(1-phenylallyl)chloridedimer, di-μ-chlorobis[2-(amino-N)[1,1′-biphenyl]-2-yl-C]dipalladium(II), di-μ-chlorobis [2-(dimethylamino)methyl]phenyl-C,N]dipalladium(II), dichloro[9,9-dimethyl-4,5-bis(diphenylphosphino)xanthene]palladium (Pd(XantPhos)Cl₂) and thereof. The base used is selected from sodium acetate, potassium acetate, lithium acetate, cesium acetate and thereof. The suitable solvent used is acetonitrile, propionitrile, isobutyronitrile and thereof;
In step-c) the catalyst used is selected from Pd(PPh₃)₄, PdCl₂, Pd(OAc)₂, Pd₂(dba)₃, Pd(PPh₃)₂Cl₂, PdCl₂(dppf), PdCl₂(dppf)·CH₂Cl₂, PdCl₂(dppp), Cyclopentadienyl allyl palladium, allylpalladium(II) chloride dimer (Pd(allyl)Cl)₂, (2-Butenyl)chloropalladium dimer, (2-Methylallyl) palladium(II) chloride dimer, palladium(1-phenylallyl)chloride dimer, di-μ-chlorobis[2-(amino-N)[1,1′-biphenyl]-2-yl-C]dipalladium(II), di-μ-chloro bis[2-(dimethylamino)methyl]phenyl-C,N]dipalladium(II), dichloro [9,9-dimethyl-4,5-bis(diphenylphosphino)xanthene]palladium (Pd(XantPhos) Cl₂) and thereof. The base used is selected from sodium carbonate, potassium carbonate, lithium carbonate, cesium carbonate and thereof; The suitable solvent used is selected from acetonitrile, propionitrile, isobutyronitrile or mixture thereof.

7. A process for the preparation of compound of Formula-XI, 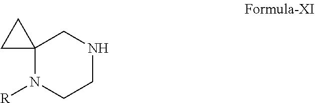
wherein, R is amino protecting group other than Boc (tert-butoxy carbonyl);
comprising the steps of:
a) reacting a compound of Formula-XII, 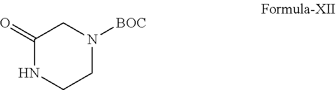 [SMILES: CC(C)(C)OC(=O)N1CCNC(=O)C1]
with benzyl bromide in presence of a base in a suitable solvent to obtain compound of Formula-XIII, 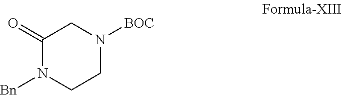 [SMILES: CC(C)(C)OC(=O)N1CCN(Cc2ccccc2)C(=O)C1]
b) treating the compound of Formula-XIII with ethyl magnesium bromide in presence of a catalyst in a suitable solvent to obtain compound of Formula-XIV, 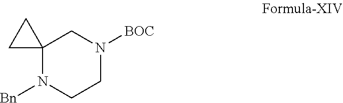 [SMILES: CC(C)(C)OC(=O)N1CCN(Cc2ccccc2)C2(CC2)C1]
c) selectively deprotecting the compound of Formula-XIV in presence of a catalyst in a suitable solvent to obtain compound of Formula-XV, 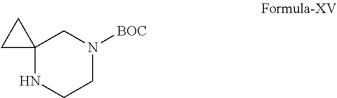 [SMILES: CC(C)(C)OC(=O)N1CCNC2(CC2)C1]
d) protecting the compound of Formula-XV in situ with amino protecting agent in presence of a base in a suitable solvent to obtain compound of Formula-XVI, 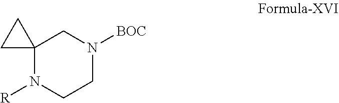
e) selectively deprotecting the compound of Formula-XVI in situ in presence of an acid in a suitable solvent to obtain compound of Formula-XI.

8. The process as claimed in claim 7, wherein,
In step-a) the base used is selected from lithium hydride, sodium hydride, potassium hydride, rubidium hydride, cesium hydride and thereof. The suitable solvent used is selected from dimethyl ether, diisopropyl ether, diethyl ether, methyl tert-butyl ether, 1,2-dimethoxyethane, tetrahydrofuran, 1,4-dioxane, monoglyme, diglyme and/or mixture thereof;
In step-b) the catalyst used is selected from ClTi(OiPr)₃, Ti(OiPr)₄, ClTi(OtBu)₃, Ti(OtBu)₄ and thereof. The suitable solvent used is selected from dimethyl ether, diisopropyl ether, diethyl ether, methyl tert-butyl ether, 1,2-dimethoxyethane, tetrahydrofuran, 1,4-dioxane, monoglyme, diglyme and/or mixtures thereof;
In step-c) the catalyst used is selected from palladium catalyst is selected from Pd(OH)₂/C, Pt, Pt/C, PtO₂, Pd, Pd/C, Rh, Ru, Ni or Raney-Ni, Zn, Sn or Fe and an acid; AlH₃—AlCl₃; hydrazine and a catalyst; [Fe₃(CO)₁₂]-methanol; hot liquid paraffin; formic acid or ammonium formate, Pd/C; LiAlH₄, NaHS, (NH₄)₂S or polysulfides and thereof; The suitable solvent used is selected from methanol, ethanol, n-propanol, isopropanol, n-butanol, isobutanol, 2-butanol, t-butanol, ethane-1,2-diol, propane-1,2-diol or mixture thereof;
In step-d) the amino protecting agent used is selected from C₁-C₆ acyl, benzyloxycarbonyl (Cbz), benzyl (Bn), benzoyl (Bz), trityl, Si(C₁-C₆ alkyl)₃, mesyl, tosyl, benzenesulfonyl, triflate and thereof. The base used is selected from “organic bases” such as methylamine, ethylamine, diisopropylamine (DIPA), diisopropylethylamine (DIPEA), diisobutylamine, triethylamine (TEA), tert-butyl amine, pyridine, 4-dimethylaminopyridine (DMAP), N-methyl morpholine (NMM), N-methylpiperidine, 1,8-diazabicyclo[5.4.0]undec-7-ene (DBU), 1,5-diazabicyclo[4.3.0]non-5-ene (DBN), 1,4-diazabicyclo[2.2.2]octane (DABCO), imidazole and thereof; The suitable solvent used is selected from “chloro solvents” such as dichloromethane, dichloroethane, chloroform, carbon tetrachloride and/or mixture thereof;
In step-e) the acid used is selected from HCl, HBr, HI and the like; The suitable solvent used is selected from methyl acetate, ethyl acetate, n-propyl acetate, isopropyl acetate, n-butyl acetate, tert-butyl acetate or the mixtures thereof.

9. A process for the preparation of compound of Formula-XIa, 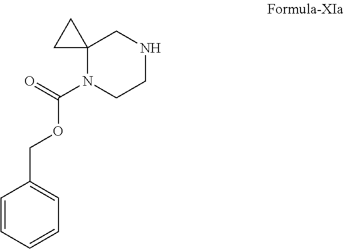 [SMILES: O=C(OCc1ccccc1)N1CCNCC12CC2]
comprising the steps of:
a) reacting a compound of Formula-XII, 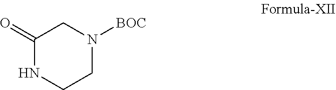 [SMILES: CC(C)(C)OC(=O)N1CCNC(=O)C1]
with benzyl bromide in presence of a base in a suitable solvent to obtain compound of Formula-XIII, 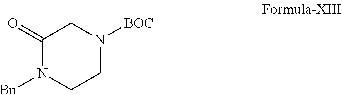 [SMILES: CC(C)(C)OC(=O)N1CCN(Cc2ccccc2)C(=O)C1]
b) treating the compound of Formula-XIII with ethyl magnesium bromide in presence of a catalyst in a suitable solvent to obtain compound of Formula-XIV, 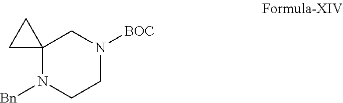 [SMILES: CC(C)(C)OC(=O)N1CCN(Cc2ccccc2)C2(CC2)C1]
c) selectively deprotecting the compound of Formula-XIV in presence of a catalyst in a suitable solvent to obtain compound of Formula-XV, 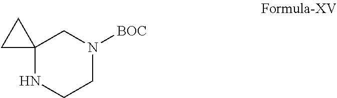 [SMILES: CC(C)(C)OC(=O)N1CCNC2(CC2)C1]
d) protecting the compound of Formula-XV in situ with benzyl chloroformate in presence of a base in a suitable solvent to obtain compound of Formula-XVIa, 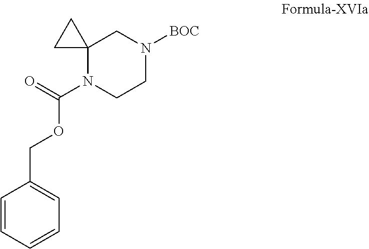 [SMILES: CC(C)(C)OC(=O)N1CCN(C(=O)OCc2ccccc2)C2(CC2)C1]
e) selectively deprotecting the compound of Formula-XVIa in situ in presence of an acid in a suitable solvent to obtain compound of Formula-XIa.

10. The process as claimed in claim 9, wherein,
In step-a) the base used is selected from lithium hydride, sodium hydride, potassium hydride, rubidium hydride, cesium hydride and thereof. The suitable solvent used is selected from dimethyl ether, diisopropyl ether, diethyl ether, methyl tert-butyl ether, 1,2-dimethoxyethane, tetrahydrofuran, 1,4-dioxane, monoglyme, diglyme and/or mixture thereof;
In step-b) the catalyst used is selected from ClTi(OiPr)₃, Ti(OiPr)₄, ClTi(OtBu)₃, Ti(OtBu)₄ and thereof. The suitable solvent used is selected from dimethyl ether, diisopropyl ether, diethyl ether, methyl tert-butyl ether, 1,2-dimethoxyethane, tetrahydrofuran, 1,4-dioxane, monoglyme, diglyme and/or mixtures thereof;
In step-c) the catalyst used is selected from palladium catalyst is selected from Pd(OH)₂/C, Pt, Pt/C, PtO₂, Pd, Pd/C, Rh, Ru, Ni or Raney-Ni, Zn, Sn or Fe and an acid; AlH₃—AlCl₃; hydrazine and a catalyst; [Fe₃(CO)₁₂]-methanol; hot liquid paraffin; formic acid or ammonium formate, Pd/C; LiAlH₄, NaHS, (NH₄)₂S or polysulfides and thereof; The suitable solvent used is selected from methanol, ethanol, n-propanol, isopropanol, n-butanol, isobutanol, 2-butanol, t-butanol, ethane-1,2-diol, propane-1,2-diol or mixture thereof;
In step-d) the base used is selected from “organic bases” such as methylamine, ethylamine, diisopropylamine (DIPA), diisopropylethylamine (DIPEA), diisobutylamine, triethylamine (TEA), tert-butyl amine, pyridine, 4-dimethylaminopyridine (DMAP), N-methyl morpholine (NMM), N-methylpiperidine, 1,8-diazabicyclo[5.4.0]undec-7-ene (DBU), 1,5-diazabicyclo[4.3.0]non-5-ene (DBN), 1,4-diazabicyclo[2.2.2]octane (DABCO), imidazole and thereof; The suitable solvent used is selected from “chloro solvents” such as dichloromethane, dichloroethane, chloroform, carbon tetrachloride and/or mixture thereof;
In step-e) the acid used is selected from HCl, HBr, HI and the like; The suitable solvent used is selected from methyl acetate, ethyl acetate, n-propyl acetate, isopropyl acetate, n-butyl acetate, tert-butyl acetate or the mixtures thereof.

11. A compound of the formulae: 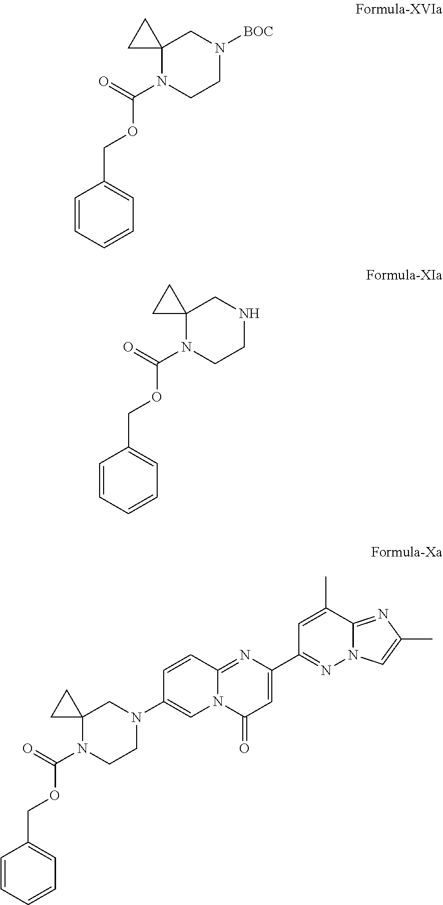 [SMILES: CC(C)(C)OC(=O)N1CCN(C(=O)OCc2ccccc2)C2(CC2)C1 ; O=C(OCc1ccccc1)N1CCNCC12CC2 ; Cc1cn2nc(-c3cc(=O)n4cc(N5CCN(C(=O)OCc6ccccc6)C6(CC6)C5)ccc4n3)cc(C)c2n1]
